(19) United States
(12) Patent Application Publication
(10) Pub. No.: US 2025/0261674 A1
(43) Pub. Date: Aug. 21, 2025

(54) PRODUCTION METHOD FOR FLAVOR COMPOSITION

(71) Applicant: TAKASAGO INTERNATIONAL CORPORATION, Tokyo (JP)

(72) Inventors: Shogo OTA, Kanagawa (JP); Ken OMORI, Kanagawa (JP); Tomoko OTA, Kanagawa (JP)

(73) Assignee: TAKASAGO INTERNATIONAL CORPORATION, Tokyo (JP)

(21) Appl. No.: 18/856,467

(22) PCT Filed: Aug. 3, 2022
(86) PCT No.: PCT/JP2022/029855

§ 371 Date: Oct. 11, 2024

(30) Foreign Application Priority Data
Apr. 15, 2022 (JP) 2022-067737

(51) Int. Cl. A23L 27/29; A23G 3/36; A23L 2/56; A61B 5/00; B01D 53/02

(52) U.S. Cl. CPC A23L 27/29; A23G 3/36; A23L 2/56; A61B 5/4011; B01D 53/025

(57) ABSTRACT

A method for producing a flavor composition is disclosed. The method includes: step (a) of preparing first flavor composition of formulation by calculating a detection rate t of aroma compounds in a retronasal aroma (RA) during eating an analyte; step (b) of calculating a detection rate 1 of aroma compounds including a RA during eating model food 1 or drinking model beverage 1 added the first flavor composition; and step (c) of preparing second flavor composition by adjusting a formulation of the first flavor composition to bring the detection rate 1 close to the detection rate t. In the method, steps (b) and (c) are repeated to prepare a final flavor composition having an aroma balance matching the detection rate t.

### TECHNICAL FIELD

The present invention relates to a method for producing a flavor composition.

### BACKGROUND ART

Due to diversification of preferences of consumers for food or beverage, there is a demand for development of food or beverage that meets the diversification of preferences. In addition, there is a demand for development of a flavor composition capable of reproducing feelings of eating and drinking real food or beverage. Along with such a trend of times, the development of a flavor composition having characteristics not present in products in the related art has been a problem.

In the related art, a flavor composition for food or beverage has been developed based on experience and sensitivity of flavorists skilled in evaluating a flavor composition with reference to results of an aroma analysis of food or beverage. As for the flavor analysis of food or beverage, an aroma concentrate is prepared by, for example, a method for collecting and concentrating aroma components floating in a headspace of food or beverage using an adsorbent, or a method for extracting aroma components from food or beverage with an organic solvent and then concentrating the aroma components by distillation treatment or the like, and is subjected to a gas chromatography analysis to obtain aroma component information.

As a common analytical technique relating to a flavor composition for food or beverage, a method for focusing on a retronasal aroma, which is aroma compounds in the exhaled breath during eating or drinking an analyte, has been known. For example, Patent Literatures 1 and 2 disclose a method for evaluating aroma expression characteristics by analyzing aroma compounds in exhaled breath using gas chromatography or the like and a method for searching for aroma components to provide a unique characterization. Patent Literature 3 proposes a method in which an aroma component remaining in saliva after drinking beverages is defined as a “lingering aroma”, and a flavor composition blended with a group of aroma components having a distinctive lingering aroma is added to impart full bodied to beverages. Patent Literature 4 discloses a flavor development method in which a flavor composition is separately added to an oral model environmental solution containing artificial saliva as a base and a non-oral environmental solution not containing artificial saliva, the volatilized aroma components are respectively collected and subjected to a component analysis, and a correction value is calculated based on the difference between the two to correct a composition ratio of the flavor composition.

### CITATION LIST

### Patent Literature

Patent Literature 1: JP2016-045156A
Patent Literature 2: JP2019-045243A
Patent Literature 3: JP2018-072210A
Patent Literature 4: JP2007-236233A

### SUMMARY OF INVENTION

### Technical Problem

However, in a method according to the related art as described above, flavored food or beverage is often greatly different from an analyte in physical properties, even if a flavor composition in which reproducing an aroma compound inherent in the analyte is prepared, it is difficult to evoke a feeling of eating or drinking the analyte during eating food or beverage added the flavor composition.

The present invention has been made in view of the above circumstances, and an object of the present invention is to provide a method for producing a flavor composition, which is capable of evoking the feeling during eating or drinking an analyte during eating or drinking food or beverage added the produced flavor composition. And the blending amount of aroma compounds can be adjusted based on scientific and objective data obtained using a person.

### Solution to Problem

As a result of diligent studies to solve the above problems, the present inventors have come up with the idea that by matching the aroma balance of retronasal aroma during eating or drinking a processed food added a flavor composition, with the aroma balance of retronasal aroma during eating or drinking a target food or beverage, it would be possible to evoke the flavor of the target food or beverage during eating or drinking a processed food added the flavor composition, and discovered that the above problems could be solved by analyzing, comparing, and blending plural aroma compounds contained in the retronasal aroma, and thus completed the present invention.

That is, the present invention relates to the following <1> to <4>.

<1> A method for producing a flavor composition, the method including:
a step (1) of analyzing a retronasal aroma during eating or drinking an analyte by gas chromatography;
a step (2) of preparing a first flavor composition containing aroma compounds, by calculating a detection rate t of these plural aroma compounds in the retronasal aroma of the step (1) based on a result of the gas chromatography analysis of the step (1);
a step (3) of preparing a model food or beverage 1 by adding the first flavor composition;
a step (4) of analyzing a retronasal aroma during eating or drinking the model food or beverage 1 by gas chromatography;
a step (5) of calculating a detection rate 1 of plural aroma compounds in the retronasal aroma of the step (4) based on a result of the gas chromatography analysis of the step (4);
a step (6) of preparing a second flavor composition by adjusting an aroma balance of the first flavor composition to bring the detection rate 1 close to the detection rate t; and
a step (7) of preparing a final flavor composition having an aroma balance matching the detection rate t by repeating the same operations as the steps (3) to (6).

<2> The method for producing a flavor composition according to <1>, wherein the step (1) includes a step of analyzing the retronasal aroma by gas chromatography/olfactometry then adding the detected aroma compound to the first flavor composition.

<3> The method for producing a flavor composition according to <2>, wherein the gas chromatography/olfactometry analysis includes a step of adjusting an odor intensity by using an aroma extract dilution analysis method.

<4> The method for producing a flavor composition according to any one of <1> to <3>, wherein the steps (1) and (4) each includes a step of adsorbing the retronasal aroma to an adsorbent and desorbing the adsorbed retronasal aroma.

### Advantageous Effects of Invention

A method for producing a flavor composition according to the present invention is a method for producing a flavor composition capable of evoking the feeling during eating or drinking an analyte. And the blending amount of aroma compounds can be adjusted based on scientific and objective data obtained using a person.

### DESCRIPTION OF EMBODIMENTS

| Hereinafter, the present invention will be described in detail, but these are examples of preferred embodiments, and the present invention is not limited to these details.

A method for producing a flavor composition according to the present invention includes the following steps (1) to (7).

### [Step (1)]

In step (1), a retronasal aroma during eating or drinking the analyte is analyzed by gas chromatography. Before or after eating or drinking the analyte, the exhaled breath of a blank is analyzed, and the result can be subtracted in step (2) described below.

Examples of the analyte include food or beverage. The food or beverage may be a solid food or a beverage, and the kind thereof is not limited. Specific examples thereof include: fruits such as peaches, apples, grapes, strawberries, mangoes, pineapples, western pears, pears, kiwi, figs, oranges, lemons, melons, watermelons, and muscats; vegetables such as carrots, cucumbers, radishes, and corns; cooked and processed meats such as steak, grilled meat, hamburger, and ham; cooked seafood such as grilled fish, steamed shellfish, and sashimi; desserts such as ice cream, ice milk, lacto ice cream, frozen desserts, yogurt, pudding, jelly, and daily desserts; confections such as caramels, candies, compressed tablet candies, crackers, biscuits, cookies, pies, chocolates, snacks, and chewing gums; Japanese style confections such as red bean pastes, steamed bun, dumpling, and Uiro; soups such as miso soup, Japanese style soup, and Western style soup; jams; spices and flavorings; various instant beverages; various instant foods; beverages such as carbonated beverages, soft beverages, fruit juice beverages, milk beverages, lactic acid bacteria beverages, energy beverages, soy milk, coffee, and tea beverages; alcoholic beverages such as beer, Chu-Hi, cocktails, sparkling wines, fruit wines, and herbal wines; stews such as beef stew and cream stew, sauces such as demi-glace sauce, oyster sauce, tomato sauce, and tartar sauce: rice, bread, pasta, curry rice, and Hayashi rice.

Specifically, the retronasal aroma is an aroma component contained in the exhaled breath escaping from the oral cavity into the nasal cavity and expelled through the anterior nostril during eating or drinking an analyte such as food or beverage, and is closely related to the flavor felt by a person during actually eating or drinking the analyte such as food or beverage.

A method for obtaining the retronasal aroma is not particularly limited, and examples thereof include a method for capturing an aroma compound expelled from a nostril using an adsorbent. At that time, the method further includes a step of adsorbing the retronasal aroma to the adsorbent and desorbing the adsorbed retronasal aroma. The desorption method is not particularly limited.

Examples of the adsorbent for aroma compounds include porous resin adsorbents such as weakly polar porous polymer beads based on 2,6-diphenyl-p-phenylene oxide.

A gas chromatography (GC) analysis is an analysis method for identifying and quantifying each component contained in an analysis target (gas such as exhaled breath), and can be performed using a general gas chromatography device. Gas chromatography/mass spectrometry (GC/MS) analyzed by a combination of a gas chromatograph device and a mass spectrometer is preferably used.

It is preferable that a gas chromatography/olfactometry (GC/O) analysis is performed on the retronasal aroma in step (1), and the detected aroma compound is contained in the first flavor composition described below.

GC/O is an analysis method in which a human nose sensation is combined with the gas chromatography analysis. Introduction of GC/O can be expected to obtain information on aroma compounds that are closer to the flavor perceive during eating or drinking the analyte. That is, a person can detect a trace amount of aroma components and adjustment is enabled by using GC/O in the case where the analyte contains a trace amount of aroma compound which is difficult to detect by gas chromatography analysis or gas chromatography/mass spectrometry.

In addition, GC/O may include a step of adjusting the odor intensity using an aroma extract dilution analysis (AEDA) method for stepwise adjusting an amount of the collected aroma compounds.

### [Step (2)]

In step (2), preparing first flavor composition by calculating a detection rate t of plural aroma compounds in the retronasal aroma of step (1) based on the result of the gas chromatography analysis of step (1).

The result of the gas chromatography analysis is obtained in the form of an integrated area of peaks of the each aroma compound in the retronasal aroma. The detection rate t of each aroma compound can be calculated based on the peak area value. The formulation of first flavor composition may also be obtained by converting the detection rate t into mass %, is not particularly limited as long as the aroma compound detected by the retronasal aroma analysis in step (1) is contained. The flavor composition was prepared by blending plural aroma compounds the ratio of the formulation.

### [Step (3)]

In step (3), model food or beverage 1 is prepared by adding the first flavor composition is prepared.

The content of the first flavor composition in the model food or beverage 1 is not particularly limited, and is, for example, 0.0001 mass % to 10 mass %, and preferably 0.001 mass % to 1.0 mass %.

In addition to the first flavor composition, the model food or beverage 1 may contain various compounds for reproducing the flavor of the analyte. A method for preparing the model food or beverage 1 is not particularly limited. The model food or beverage 1 may be a solid food or a beverage.

### [Step (4)]

In step (4), retronasal aroma during eating or drinking model food or beverage 1 is analyzed by gas chromatography. Before or after eating or drinking the model food or beverage 1, the exhaled breath of a blank is analyzed, and the result can be subtracted in step (5) described below.

That is, according to the present invention, the first flavor composition is not a final product. And then analyzing retronasal aroma during eating or drinking the model food or beverage 1.

The retronasal aroma and the gas chromatography analysis are as described in step (1).

### [Step (5)]

In step (5), a detection rate 1 of plural aroma compounds in the retronasal aroma of the step (4) is calculated based on the result of the gas chromatography analysis of the step (4).

The result of the gas chromatography analysis in step (4) is obtained in the form of a peak area of each aroma compound contained in the retronasal aroma. The detection rate 1 of each aroma compound is calculated based on the peak area value.

### [Step (6)]

In step (6), a second flavor composition is prepared by adjusting an aroma balance of the first flavor composition to bring the detection rate 1 close to the detection rate t.

In step (6), adjustment such as increasing an amount of an insufficient compound and decreasing an amount of an excessive compound in the first flavor composition is performed with reference to the detection rate t, that is, the aroma balance of the retronasal aroma derived from the analyte. The food or beverage added second flavor composition obtained through such adjustment has flavor closer to the original analyte than the food or beverage added the first flavor composition.

### [Step (7)]

In step (7), a final flavor composition having an aroma balance matching the detection rate t is prepared by repeating the same operation as steps (3) to (6).

The same operation as steps (3) to (6) was repeated to prepare the third flavor composition, then the fourth flavor composition, the fifth flavor composition, and so on. When the aroma balance of retronasal aroma during eating or drinking model food or beverage match the aroma balance of step (1), that flavor composition is as the final flavor composition.

In the case where the aroma balance of the second flavor composition matches it of the retronasal aroma derived from the analyte, that process is ended. In addition, the “match” in step (7) does not only mean a perfect match, but also includes a match between the flavor of the object to be analyzed and the flavor of the final flavored food or beverage to a degree that would be acceptable to an average consumer.

In this way, it is possible to create a flavor composition that evokes the analyte even when added to food or beverage with different matrices, without relying too much on the sense of the flavorist.

The flavor composition produced by the method for producing a flavor composition according to the present invention and food or beverage containing the flavor composition are produced after objectively evaluating whether the flavor balance when a target analyte is eaten or drunk can be reproduced. With this method, even if one is not a skilled flavorist, it is relatively easy to reproduce the flavor of the analyte in food or beverage with different matrices (processed food or beverage).

### EXAMPLES

Next, the present invention will be described in more detail with reference to Examples, but the present invention is not limited to these Examples.

### [Example 1] Production of Apple Flavor Composition for Beverages

### <Collection of Exhaled Breath During Eating Apples>

Immediately before aroma collection, an internal standard substance (Nonan-5-one) was added to the aroma compound adsorbent (TENAX TA: manufactured by GERSTEL K.K.), and the exhaled breath during eating a bite-sized apple and swallowing it, was sucked using an air pump and adsorbed into the aroma compound adsorbent. The exhaled breath was collected three times for GC/MS and once for GC/O. In addition, the aroma in exhaled breath as a blank was collected in a state in which nothing was put in the oral cavity for the same time as the apple eating time. The exhaled breath was collected once for GC/MS and once for GC/O.

### <Aroma Analysis by GC/MS of the Exhaled Breath During Eating Apples>

The aroma component adsorbent was purged with nitrogen (100 mL/min, 30 minutes), and then subjected to desorption by heating with a thermal desorption unit (TDU). Then, the desorbed aroma components were introduced into a gas chromatograph (“GC6890NGC” manufactured by Agilent Technologies International Japan, Ltd.) equipped with a mass spectrometer, and peak area values of each aroma component were obtained from GC/MS analysis of exhaled breath during eating apples and exhaled breath of blank.

### [Measurement Device Conditions]

TDU (Thermal Desorption Unit)
Desorption temperature: 250° C.
Desorption time: 5 min
Temperature program: 30° C. to 250° C., 720° C./min
CIS (Cooled Injection System)
Trap temperature: 10° C.
Temperature program: 10° C. to 150° C., 960° C./min, 150° C. to 250° C., 720° C./min (held for 15 min)
GC (Gas Chromatography)
Column: BC-WAX (manufactured by GL Sciences Inc., 0.25 mm i.d.×50 m, film thickness: 0.15 μm)
Oven program: 40° C. to 200° C., 4° C./min, 200° C. to 230° C., 20° C./min
Detector: MS (“5977A” manufactured by Agilent Technologies International Japan, Ltd.)
Detector MS (“5977A” manufactured by Agilent Technologies International Japan, Ltd.)

### <Aroma Analysis by GC/O of Exhaled Breath During Eating Apples>

The aroma compound adsorbent was purged with nitrogen (100 mL/min, 30 minutes), then the aroma compounds were introduced into a gas chromatograph equipped with a GC-O port with a portable thermal desorber “Handy TD TD265” manufactured by GL Science Inc., and exhaled breath during eating apples and exhaled breath of blank was analyzed by GC/O. As a result, there was no aroma component detected only by the GC/O analysis.

### [Measurement Device Conditions]

TDU (Thermal Desorption Unit)
Desorption temperature: 250° C.
Desorption time: 1.5 min
Temperature program: 40° C. to 250° C., 2700° C./min
CIS (Cooled Injection System)
Trap temperature: 10° C.
Temperature program: 10° C. to 150° C., 960° C./min, 150° C. to 250° C., 720° C./min (held for 15 min)
GC (Gas Chromatography)
Column: BC-WAX (manufactured by GL Sciences Inc., 0.25 mm i.d.×50 m, film thickness: 0.15 μm)
Oven program: 70° C. to 230° C.
Detector: FID (“7980A” manufactured by Agilent Technologies International Japan, Ltd.)
Detector FID (“7980A” manufactured by Agilent Technologies International Japan, Ltd.)

### <Preparation of First Flavor Composition (Apple Flavor A)>

An internal standard ratio was calculated based on an area value of each aroma compound, which was detected by the GC/MS analysis of each compound in the exhaled breath during eating apples, and an area value of Nonan-5-one used as the internal standard substance. Next, an internal standard ratio was calculated in the same manner for exhaled breath of the blank, and was subtracted from the internal standard ratio during eating apples. The value of each compound stand for the internal standard ratio of the retronasal aroma during eating apples. Next, a detection rate t with the total being 100% was calculated based on the internal standard ratio. Thereafter the detection rate t was converted into mass %, and the aroma component was diluted with a solvent to obtain the formulation of the first flavor composition (apple flavor A). Apple flavor A was prepared by blending plural aroma compounds the ratio of the formulation shown in Table 1.

### <Preparation of Model Apple Beverage 1>

The sugar and organic acid analysis of commercially available apples purchased in the season was performed, and beverage base having an adjusted the ratio sugar and organic acid was prepared based on the result. Model apple beverage 1 was prepared by adding apple flavor A to the above beverage base with 0.1 mass %. In addition, beverage not added flavor composition was prepared for blank measurement.

### <Collection of Exhaled Breath During Drinking Model Apple Beverage 1>

Exhaled breath during drinking model apple beverage 1 and blank beverage were collected by the same method as in the case of eating apples. Exhaled breath during drinking model apple beverage 1 collected three times for GC/MS. In addition, the exhale breath during drinking blank beverage collected once for GC/MS.

### <Aroma Analysis by GC/MS of Exhaled Breath During Drinking Model Apple Beverage 1>

Exhaled breath during drinking model apple beverage 1 and blank beverage was analyzed by GC/MS according to the same method and analysis conditions as those for exhaled breath during eating apples.

### <Calculation of Detection Rate 1>

Detection rate 1 with the total being 100% was calculated based on an internal standard ratio of the retronasal aroma during drinking the model apple beverage 1, which was calculated by the same method as in <Preparation of first flavor composition (apple flavor A)>.

### <Detection Rate Comparison and Sensory Evaluation>

As shown in Table 1, the aroma balance was significantly different when the detection rate t of the retronasal aroma during eating apples was compared with the detection rate 1 of the retronasal aroma during drinking model apple beverage 1.

In addition, as a result of drinking model apple beverage 1, the fresh flavor felt during eating apples was insufficient.

### <Preparation of Second Flavor Composition (Apple Flavor B)>

Formulation values of apple flavor B were adjusted in order to bring the detection rate 1 close to the detection rate t. Calibration curve was created based on the internal standard ratio value of the retronasal aroma during drinking model apple beverage 1 and the formulation value of apple flavor A. Then, using it, formulation value of each aroma compound of apple flavor B was calculated based on the internal standard ratio value of the retronasal aroma during eating apples. Apple flavor B was prepared by blending plural aroma compounds the ratio of the formulation shown in Table 1.

### <Preparation of Model Apple Beverage 2>

A model apple beverage 2 was prepared by adding apple flavor B to the above beverage base with 0.1 mass %. In addition, beverage not added flavor composition was prepared for blank measurement.

### <Collection of Exhaled Breath During Drinking Model Apple Beverage 2>

Exhaled breath during drinking model apple beverage 2 and blank beverage was collected according to the same method as in the case of model apple beverage 1.

### <Aroma Analysis by GC/MS of Exhaled Breath During Drinking Model Apple Beverage 2>

Exhaled breath during drinking model apple beverage 2 and blank beverage was analyzed by GC/MS according to the same method and analysis conditions as those for exhaled breath during drinking model apple beverage 1.

### <Calculation of Detection Rate 2>

Detection rate 2 with the total being 100% was calculated based on an internal standard ratio of the retronasal aroma during drinking model apple beverage 2, which was calculated by the same method as in the case of detection rate 1.

### <Detection Rate Comparison and Sensory Evaluation>

As shown in Table 1, the aroma balance was slightly different when making a comparison detection rate 2 and detection rate t.

In addition, as a result of drinking model apple beverage 2, the fresh flavor along with sweetness during eating apples was increased as compared with the model apple beverage 1, but was still insufficient.

### <Preparation of Third Flavor Composition (Apple Flavor C)>

Formulation value of apple flavor C was adjusted in order to bring the detection rate 2 close to the detection rate t. Calibration curve was created based on the internal standard ratio value of the retronasal aroma during drinking model apple beverage 1 and 2, and the formulation value of each flavor composition. Then, using it, formulation value of each aroma compound of apple flavor C was calculated based on the internal standard ratio value of the retronasal aroma during eating apples. Apple flavor C was prepared by blending plural aroma compounds the ratio of the formulation shown in Table 1.

### <Preparation of Model Apple Beverage 3>

Model apple beverage 3 was prepared by adding apple flavor C to the above beverage base with 0.1 mass %. In addition, beverage not added flavor composition was prepared for blank measurement.

### <Collection of Exhaled Breath During Drinking Model Apple Beverage 3>

Exhaled breath during drinking model apple beverage 3 and blank beverage was collected according to the same method as in the case of model apple beverage 1.

### <Aroma Analysis by GC/MS of Exhaled Breath During Drinking Model Apple Beverage 3>

Exhaled breath during drinking model apple beverage 3 and blank beverage were analyzed by GC/MS according to the same method and analysis conditions as those for exhaled breath during drinking model apple beverage 1.

### <Calculation of Detection Rate 3>

Detection rate 3 with the total being 100% was calculated based on an internal standard ratio of the retronasal aroma during drinking model apple beverage 3, which was calculated by the same method as in the case of detection rate 1.

### <Detection Rate Comparison and Sensory Evaluation>

As shown in Table 1, the aroma balance was similar when making a comparison detection rate 3 and detection rate t.

In addition, as a result of drinking model apple beverage 3, the fresh and juicy flavor with sweetness during eating apples was evoked.

### <Sensory Evaluation by Trained Panelists>

First, four trained panelists performed sensory evaluation on the flavor during eating apples. Next, they performed sensory evaluation on the flavor during drinking model apple beverage 1 and 3. Each model apple beverage was provided to panelists in a plastic cup with a different three-digit random number written on it. Although panelists were not informed of which was the model apple beverage 1 or 3, as a result, all panelists answered that model apple beverage 3 evoked the flavor felt during eating apples as compared with model apple beverage 1.

TABLE 1
List of calculated values in flavor preparation process according to retronasal
aroma analysis during eating apples and drinking apple beverages

| | | | | Retronasal aroma during drinking | |
|---|---|---|---|---|---|
| | Retronasal aroma during eating apples | | | model apple beverage 1 | |
| | Internal | | Apple | Internal | |
| Aroma | standard | Detection | flavor A | standard | Detection |
| compounds | ratio | rate t | Formulation | ratio | rate 1 |
| Apple-1 | 22,975 | 31 | 3.5 | 70,589 | 45 |
| Apple-2 | 8,214 | 11 | 1.3 | 19,468 | 13 |
| Apple-3 | 7,562 | 10 | 0.85 | 5,604 | 3.6 |
| Apple-4 | 7,068 | 9.6 | 0.60 | 2,238 | 1.4 |
| Apple-5 | 6,640 | 9.0 | 0.76 | 19,655 | 13 |
| Apple-6 | 2,817 | 3.8 | 0.30 | 2,571 | 1.7 |
| Apple-7 | 2,496 | 3.4 | 0.28 | 4,570 | 2.9 |
| Apple-8 | 1,962 | 2.7 | 0.12 | 3,435 | 2.2 |
| Apple-9 | 1,577 | 2.1 | 0.26 | 3,323 | 2.1 |
| Apple-10 | 1,323 | 1.8 | 0.070 | 1,327 | 0.86 |
| Apple-11 | 1,273 | 1.7 | 0.080 | 1,695 | 1.1 |
| Apple-12 | 1,094 | 1.5 | 0.18 | 3,215 | 2.1 |
| Apple-13 | 1,075 | 1.5 | 0.25 | 499 | 0.32 |
| Apple-14 | 845 | 1.1 | 0.10 | 1,539 | 0.99 |
| Apple-15 | 843 | 1.1 | 0.10 | 2,450 | 1.6 |
| Apple-16 | 615 | 0.83 | 0.080 | 1,961 | 1.3 |
| Apple-17 | 615 | 0.83 | 0.10 | 1,344 | 0.87 |
| Apple-18 | 574 | 0.78 | 0.090 | 2,629 | 1.7 |
| Apple-19 | 536 | 0.73 | 0.090 | 1,767 | 1.1 |
| Apple-20 | 513 | 0.70 | 0.10 | 209 | 0.13 |
| Apple-21 | 408 | 0.55 | 0.090 | 1,351 | 0.87 |
| Apple-22 | 291 | 0.39 | 0.040 | | |
| Apple-23 | 230 | 0.31 | 0.010 | 183 | 0.12 |
| Apple-24 | 169 | 0.23 | 0.033 | 666 | 0.43 |
| Apple-25 | 100 | 0.14 | 0.014 | 269 | 0.17 |
| Apple-26 | 83 | 0.11 | 0.0060 | 20 | 0.013 |
| Apple-27 | 71 | 0.10 | 0.010 | 194 | 0.13 |
| Apple-28 | 67 | 0.090 | 0.0080 | 28 | 0.018 |
| Apple-29 | 21 | 0.029 | 0.0080 | 121 | 0.078 |
| Apple-30 | 19 | 0.026 | 0.010 | 119 | 0.077 |
| Apple-others | 1,624 | 2.2 | 0.40 | 2,167 | 1.4 |
| Water-ethanol mixed | | | Remainder | | |
| solution | | | | | |

| | | Retronasal aroma during drinking | | | Retronasal aroma during drinking | |
|---|---|---|---|---|---|---|
| | | model apple beverage 2 | | | model apple beverage 3 | |
| | Apple | Internal | | Apple | Internal | |
| Aroma | flavor B | standard | Detection | flavor C | standard | Detection |
| compounds | Formulation | ratio | rate 2 | Formulation | ratio | rate 3 |
| Apple-1 | 1.2 | 34,962 | 34 | 0.90 | 22,156 | 30 |
| Apple-2 | 0.60 | 12,354 | 12 | 0.50 | 8,585 | 11 |
| Apple-3 | 1.2 | 6,724 | 6.6 | 1.4 | 6,313 | 8.5 |
| Apple-4 | 3.0 | 10,564 | 10 | 2.1 | 7,150 | 9.6 |
| Apple-5 | 0.30 | 10,713 | 10 | 0.20 | 5,857 | 7.8 |
| Apple-6 | 0.30 | 2,742 | 2.7 | 0.32 | 2,145 | 2.9 |
| Apple-7 | 0.14 | 3,423 | 3.3 | 0.12 | 2,327 | 3.1 |
| Apple-8 | 0.10 | 1,786 | 1.7 | 0.10 | 3,184 | 4.3 |
| Apple-9 | 0.13 | 2,102 | 2.1 | 0.12 | 1,766 | 2.4 |
| Apple-10 | 0.090 | 2,381 | 2.3 | 0.060 | 1,246 | 1.7 |
| Apple-11 | 0.080 | 2,190 | 2.1 | 0.050 | 1,195 | 1.6 |
| Apple-12 | 0.060 | 1,407 | 1.4 | 0.050 | 920 | 1.2 |
| Apple-13 | 0.50 | 1,322 | 1.3 | 0.42 | 795 | 1.1 |
| Apple-14 | 0.060 | 1,325 | 1.3 | 0.040 | 717 | 0.96 |
| Apple-15 | 0.040 | 1,409 | 1.4 | 0.030 | 821 | 1.1 |
| Apple-16 | 0.040 | 1,308 | 1.3 | 0.020 | 589 | 0.79 |
| Apple-17 | 0.060 | 1,090 | 1.1 | 0.040 | 640 | 0.86 |
| Apple-18 | 0.020 | 594 | 0.58 | 0.030 | 735 | 0.99 |
| Apple-19 | 0.030 | 783 | 0.77 | 0.030 | 613 | 0.82 |
| Apple-20 | 0.24 | 681 | 0.67 | 0.20 | 636 | 0.85 |
| Apple-21 | 0.030 | 605 | 0.59 | 0.030 | 517 | 0.69 |
| Apple-22 | 0.020 | | | 0.040 | 453 | 0.61 |
| Apple-23 | 0.010 | 224 | 0.22 | 0.010 | 186 | 0.25 |
| Apple-24 | 0.0080 | 202 | 0.20 | 0.0080 | 152 | 0.20 |
| Apple-25 | 0.0050 | 150 | 0.15 | 0.0040 | 94 | 0.13 |
| Apple-26 | 0.020 | 20 | 0.020 | 0.080 | 7 | 0.010 |
| Apple-27 | 0.0040 | 91 | 0.089 | 0.0040 | 71 | 0.10 |
| Apple-28 | 0.020 | 89 | 0.087 | 0.020 | 54 | 0.072 |
| Apple-29 | 0.0030 | 158 | 0.15 | 0.00040 | 9 | 0.012 |
| Apple-30 | 0.0020 | 40 | 0.039 | 0.0020 | 30 | 0.041 |
| Apple-others | 0.98 | 895 | 0.874 | 2.2 | 4,697 | 6.3 |
| Water-ethanol mixed | Remainder | | | Remainder | | |
| solution | | | | | | |

### [Example 2] Production of Grape Flavor Composition for Beverages

### <Collection of Exhaled Breath During Eating Grapes>

Exhaled breath during eating grapes was collected by the same method for apples.

### <Aroma Analysis by GC/MS of Exhaled Breath During Eating Grapes>

Exhaled breath during eating grapes and exhaled breath of blank were analyzed by GC/MS according to the same method and analysis conditions as those for exhaled breath during eating apples.

### <Aroma Analysis by GC/O of Exhaled Breath During Eating Grapes>

Exhaled breath during eating grapes and exhaled breath of blank were analyzed by GC/O according to the same method and analysis conditions as those for exhaled breath during eating apples. As a result, it was confirmed the presence and its odor intensity of compound that was not detected by GC/MS in exhaled breath during eating grapes.

### <Preparation of First Flavor Composition (Grape Flavor A)>

An internal standard ratio of a retronasal aroma during eating grapes was calculated by the same method as in the case of apples, and then, the detection rate t with the total being 100% was calculated based on the internal standard ratio. Thereafter, the detection rate t was converted to mass %, the compound detected only by GC/O was added thereto, and the mixture was further appropriately diluted with a solvent to obtain a formulation of first flavor composition (grape flavor A). Formulation value of the compound detected only by GC/O was the same as the lowest formulation value among the compound detected by GC/MS. Grape flavor A was prepared by blending plural aroma compounds the ratio of the formulation shown in Table 2.

### <Preparation of Model Grape Beverage 1>

The sugar and organic acid analysis of commercially available grapes purchased in the season was performed, and beverage base having an adjusted the ratio of sugar and organic acid was prepared based on the result. Model grape beverage 1 was prepared by adding grape flavor A to the above beverage base with 0.1 mass %. In addition, beverage not added flavor composition was prepared for blank measurement.

### <Collection of Exhaled Breath During Drinking Model Grape Beverage 1>

Exhaled breath during drinking model grape beverage 1 was collected three times for GC/MS, and collected once for GC/O according to the same method as in the case of model apple beverage 1. In addition, the exhaled breath during drinking blank beverage was collected once for GC/MS and once for GC/O according to the same method.

### <Aroma Analysis by GC/MS of Exhaled Breath During Drinking Model Grape Beverage 1>

Exhaled breath during drinking model grape beverage 1 and blank beverage was analyzed by GC/MS according to the same method and analysis conditions as those for exhaled breath during eating grapes.

### <Aroma Analysis by GC/O of Exhaled Breath During Drinking Model Grape Beverage 1>

Exhaled breath during drinking model grape beverage 1 and blank beverage was analyzed by GC/O according to the same method and analysis conditions as those for exhaled breath during eating grapes. As a result, it was confirmed the presence that compound not detected by GC/MS in exhaled breath during drinking model grape beverage 1 and its odor intensity was equivalent to that of exhaled breath during eating grapes.

### <Calculation of Detection Rate 1>

Detection rate 1 with the total being 100% was calculated based on an internal standard ratio of the retronasal aroma during drinking the model grape beverage 1, which was calculated by the same method as in <Preparation of first flavor composition (grape flavor A)>.

### <Detection Rate Comparison and Sensory Evaluation>

As shown in Table 2, the aroma balance was significantly different when the detection rate t of the retronasal aroma during eating grapes was compared with the detection rate 1 of the retronasal aroma during drinking the model grape beverage 1.

In addition, as a result of drinking the model grape beverage 1, the fresh flavor felt during eating grapes was insufficient.

### <Preparation of Second Flavor Composition (Grape Flavor B)>

Formulation values of grape flavor B were adjusted in order to bring the detection rate 1 close to the detection rate t. Calibration curve was created based on the internal standard ratio value of the retronasal aroma during drinking model grape beverage 1 and the formulation value of grape flavor A. Then, using it, formulation value of each aroma compound of grape flavor B was calculated based on the internal standard ratio value of the retronasal aroma during eating grapes. Formulation value of the compound detected only by GC/O because of evaluated that the odor intensity of retronasal aroma during drinking model grape beverage 1 and retronasal aroma during eating grapes was equivalent, was set to the same formulation value as grape flavor A. Grape flavor B was prepared by blending plural aroma compounds the ratio of the formulation shown in Table 2.

### <Preparation of Model Grape Beverage 2>

A model grape beverage 2 was prepared by adding grape flavor B to the above beverage base with 0.1 mass %. In addition, beverage not added flavor composition was prepared for blank measurement.

### <Collection of Exhaled Breath During Drinking Model Grape Beverage 2>

Exhaled breath during drinking model grape beverage 2 and blank beverage was collected according to the same method as in the case of model grape beverage 1.

### <Aroma Analysis by GC/MS of Exhaled Breath During Drinking Model Grape Beverage 2>

Exhaled breath during drinking model grape beverage 2 and blank beverage was analyzed by GC/MS according to the same method and analysis conditions as those for exhaled breath during drinking model grape beverage 1.

### <Aroma Analysis by GC/O of Exhaled Breath During Drinking Model Grape Beverage 2>

Exhaled breath during drinking model grape beverage 2 and blank beverage was analyzed by GC/O according to the same method and analysis conditions as those for exhaled breath during drinking model grape beverage 1. As a result, it was confirmed that aroma compound not detected by GC/MS in the exhaled breath during drinking model grape beverage 2 and its odor intensity was equivalent to that of exhaled breath during eating grapes.

### <Calculation of Detection Rate 2>

Detection rate 2 with the total being 100% was calculated based on an internal standard ratio of the retronasal aroma during drinking the model grape beverage 2, which was calculated by the same method as in the case of detection rate 1.

### <Detection Rate Comparison and Sensory Evaluation>

As shown in Table 2, the aroma balance was slightly different when making a comparison detection rate 2 and detection rate t.

In addition, as a result of drinking model grape beverage 2, the fresh flavor along with sweetness during eating grapes was increased as compared with model grape beverage 1, but was still insufficient.

### <Preparation of Third Flavor Composition (Grape Flavor C)>

Formulation value of grape flavor C was adjusted in order to bring the detection rate 2 close to the detection rate t. Calibration curve was created based on the internal standard ratio value of the retronasal aroma during drinking model grape beverage 1 and 2, and the formulation value of each flavor composition. Then, using it, formulation value of each aroma compound of grape flavor C was calculated based on the internal standard ratio value of the retronasal aroma during eating grapes. Formulation value of aroma compound detected only by GC/O because of evaluated that the odor intensity of ratronasal aroma during drinking model grape beverage 2 and retronasal aroma during eating grapes was equivalent, was set to the same formulation value as grape flavor B. Grape flavor C was prepared by blending plural aroma compounds the ratio of the formulation shown in Table 2.

### <Preparation of Model Grape Beverage 3>

Model grape beverage 3 was prepared by adding grape flavor C to the above beverage base with 0.1 mass %. In addition, beverage not added flavor composition was prepared for blank measurement.

### <Collection of Exhaled Breath During Drinking Model Grape Beverage 3>

Exhaled breath during drinking model grape beverage 3 and blank beverage was collected according to the same method as in the case of model grape beverage 1.

### <Aroma Analysis by GC/MS of Exhaled Breath During Drinking Model Grape Beverage 3>

Exhaled breath during drinking model grape beverage 3 and blank beverage was analyzed by GC/MS according to the same method and analysis conditions as those for exhaled breath during drinking model grape beverage 1.

### <Aroma Analysis by GC/O of Exhaled Breath During Drinking Model Grape Beverage 3>

Exhaled breath during drinking model grape beverage 3 and blank beverage was analyzed by GC/O according to the same method and analysis conditions as those for exhaled breath during drinking model grape beverage 1. As a result, it was confirmed the presence that aroma compound not detected by GC/MS in the exhaled breath during drinking model grape beverage 3 and its odor intensity was equivalent to that of exhaled breath during eating grapes.

### <Calculation of Detection Rate 3>

Detection rate 3 with the total being 100% was calculated based on an internal standard ratio of the retronasal aroma during drinking model grape beverage 3, which was calculated by the same method as in the case of detection rate 1.

### <Detection Rate Comparison and Sensory Evaluation>

As shown in Table 2, the aroma balance was similar when making a comparison detection rate 3 and detection rate t.

In addition, as a result of drinking model grape beverage 3, the fresh and juicy flavor with fiber feels and sweetness felt during eating grapes was evoked.

### <Sensory Evaluation by Trained Panelists>

First, four trained panelists performed sensory evaluation on the flavor during eating grapes. Next, they performed sensory evaluation on the flavor during drinking model grape beverage 1 and 3. Each model grape beverage was provided to panelists in a plastic cup with a different three-digit random number written on it. Although panelists were not informed of which was model grape beverage 1 or 3, as a result, all panelists answered that model grape beverage 3 evoked the flavor felt during eating grapes as compared with model grape beverage 1.

TABLE 2
List of calculated values in flavor preparation process according to retronasal
aroma analysis during eating grapes and drinking grape beverages

| | Retronasal aroma during eating | | | Retronasal aroma during drinking | |
|---|---|---|---|---|---|
| | grapes | | | model grape beverage 1 | |
| | Internal | | Grape | Internal | |
| Aroma | standard | Detection | flavor A | standard | Detection |
| compounds | ratio | rate t | Formulation | ratio | rate 1 |
| Grape-1 | 205,947 | 93 | 37.3 | 163,775 | 82 |
| Grape-2 | 2,869 | 1.3 | 0.52 | 9,495 | 4.8 |
| Grape-3 | 2,796 | 1.3 | 0.50 | 10,973 | 5.5 |
| Grape-4 | 886 | 0.40 | 0.16 | 58 | 0.029 |
| Grape-5 | 777 | 0.35 | 0.14 | 2,643 | 1.3 |
| Grape-6 | 650 | 0.29 | 0.12 | 3,015 | 1.5 |
| Grape-7 | 589 | 0.27 | 0.11 | 1,896 | 0.95 |
| Grape-8 | 556 | 0.25 | 0.10 | 576 | 0.29 |
| Grape-9 | 519 | 0.24 | 0.10 | 176 | 0.088 |
| Grape-10 | 491 | 0.22 | 0.090 | 567 | 0.28 |
| Grape-11 | 360 | 0.16 | 0.070 | 479 | 0.24 |
| Grape-12 | 277 | 0.13 | 0.050 | 2,056 | 1.0 |
| Grape-13 | 270 | 0.12 | 0.050 | 176 | 0.088 |
| Grape-14 | 214 | 0.10 | 0.0040 | 55 | 0.028 |
| Grape-15 | 204 | 0.093 | 0.040 | 887 | 0.45 |
| Grape-16 | 110 | 0.050 | 0.020 | 559 | 0.28 |
| Grape-17 | 85 | 0.039 | 0.015 | 4 | 0.0018 |
| Grape-18 | 79 | 0.036 | 0.014 | 212 | 0.11 |
| Grape-19 | 74 | 0.033 | 0.013 | | |
| Grape-20 | 58 | 0.026 | 0.010 | 222 | 0.11 |
| Grape-21 | 53 | 0.024 | 0.010 | 187 | 0.094 |
| Grape-22 | 53 | 0.024 | 0.010 | 374 | 0.19 |
| Grape-others | 2,794 | 1.3 | 0.52 | 865 | 0.43 |
| Water-ethanol mixed | | | Remainder | | |
| solution | | | | | |

| | | Retronasal aroma during | | | Retronasal aroma during | |
|---|---|---|---|---|---|---|
| | | drinking model grape beverage 2 | | | drinking model grape beverage 3 | |
| | Grape | Internal | | Grape | Internal | |
| Aroma | flavor B | standard | Detection | flavor C | standard | Detection |
| compounds | Formulation | ratio | rate 2 | Formulation | ratio | rate 3 |
| Grape-1 | 42.4 | 143,631 | 87 | 36 | 143,194 | 94 |
| Grape-2 | 0.35 | 4,660 | 2.8 | 0.12 | 1,452 | 0.95 |
| Grape-3 | 0.24 | 7,603 | 4.6 | 0.060 | 1,961 | 1.3 |
| Grape-4 | 0.80 | 201 | 0.12 | 2.1 | 637 | 0.42 |
| Grape-5 | 0.080 | 1,502 | 0.91 | 0.024 | 547 | 0.36 |
| Grape-6 | 0.050 | 837 | 0.51 | 0.024 | 629 | 0.41 |
| Grape-7 | 0.060 | 674 | 0.41 | 0.030 | 478 | 0.31 |
| Grape-8 | 0.10 | 964 | 0.58 | 0.036 | 858 | 0.56 |
| Grape-9 | 0.50 | 364 | 0.22 | 0.36 | 317 | 0.21 |
| Grape-10 | 0.10 | 698 | 0.42 | 0.048 | 343 | 0.23 |
| Grape-11 | 0.080 | 395 | 0.24 | 0.048 | 202 | 0.13 |
| Grape-12 | 0.010 | 348 | 0.21 | 0.0048 | 247 | 0.16 |
| Grape-13 | 0.12 | 469 | 0.28 | 0.048 | 354 | 0.23 |
| Grape-14 | 0.020 | 68 | 0.041 | 0.030 | 107 | 0.071 |
| Grape-15 | 0.020 | 523 | 0.32 | 0.0048 | 149 | 0.10 |
| Grape-16 | 0.0080 | 319 | 0.19 | 0.0024 | 121 | 0.079 |
| Grape-17 | 0.12 | 300 | 0.18 | 0.024 | 96 | 0.063 |
| Grape-18 | 0.0080 | 127 | 0.077 | 0.0030 | 62 | 0.041 |
| Grape-19 | 0.080 | | | 0.12 | 50 | 0.033 |
| Grape-20 | 0.010 | 206 | 0.13 | 0.0018 | 89 | 0.058 |
| Grape-21 | 0.010 | 216 | 0.13 | 0.0018 | 46 | 0.030 |
| Grape-22 | 0.0040 | 270 | 0.16 | 0.0006 | 87 | 0.057 |
| Grape-others | 1.1 | 508 | 0.31 | 0.55 | 191 | 0.13 |
| Water-ethanol mixed | Remainder | | | Remainder | | |
| solution | | | | | | |

### [Example 3] Production of Peach Flavor Composition for Beverages

### <Collection of Exhaled Breath During Eating Peaches>

Exhaled breath during eating peaches was collected by the same method for apples.

### <Aroma Analysis by GC/MS of Exhaled Breath During Eating Peaches>

Exhaled breath during eating peaches and exhaled breath of blank was analyzed by GC/MS according to the same method and analysis conditions as those for the exhaled breath during eating apples.

### <Aroma Analysis by GC/O of Exhaled Breath During Eating Peaches>

Exhaled breath during eating peaches and exhaled breath of a blank was analyzed by GC/O according to the same method and analysis conditions as apples. As a result, it was confirmed the presence and its odor intensity of the aroma compound that was not detected by GC/MS in the exhaled breath during eating peaches.

### <Preparation of First Flavor Composition (Peach Flavor A)>

An internal standard ratio of retronasal aroma during eating peaches by the same method as in the case of apples, and then, the detection rate t with the total being 100% was calculated based on the internal standard ratio. Thereafter, the detection rate t was converted to mass %, the compound detected only by GC/O was added thereto, and the mixture was further appropriately diluted with a solvent to obtain a formulation of first flavor composition (peach flavor A). Formulation value of the compound detected only by GC/O was the same as the lowest formulation value among the compound detected by GC/MS. Peach flavor A was prepared by blending each aroma compound the ratio of the formulation shown in Table 3.

### <Preparation of Model Peach Beverage 1>

The sugar and organic acid analysis of commercially available peaches purchased in the season was performed, and beverage base having an adjusted the ratio of sugar and organic acid was prepared based on the result. Model peach beverage 1 was prepared by adding peach flavor A to the above beverage base with 0.1 mass %. In addition, beverage not added flavor composition was prepared for blank measurement.

### <Collection of the Exhaled Breath During Drinking Model Peach Beverage 1>

Exhaled breath during drinking model peach beverage 1 and blank beverage were collected according to the same method as in the case of model grape beverage 1.

### <Aroma Analysis by GC/MS of Exhaled Breath During Drinking Model Peach Beverage 1>

Exhaled breath during drinking model peach beverage 1 and blank beverage were analyzed by GC/MS according to the same method and analysis conditions as those for the exhaled breath during eating peaches.

### <Aroma Analysis by GC/O of Exhaled Breath During Drinking Model Peach Beverage 1>

Exhaled breath during drinking model peach beverage 1 and blank beverage were analyzed by GC/O according to the same method and analysis conditions as those of exhaled breath during eating peaches. As a result, it was confirmed the presence that aroma compound not detected by GC/MS in the exhaled breath during drinking model peach beverage 1 and its odor intensity was equivalent to that of exhaled breath during eating peaches.

### <Calculation of Detection Rate 1>

Detection rate 1 with the total being 100% was calculated based on an internal standard ratio of the retronasal aroma during drinking model peach beverage 1, which was calculated by the same method as in <Preparation of first flavor composition (peach flavor A)>.

### <Detection Rate Comparison and Sensory Evaluation>

As shown in Table 3, the aroma balance was significantly different when the detection rate t of the retronasal aroma during eating peaches was compared with the detection rate 1 of the retronasal aroma during drinking model peach beverage 1.

In addition, as a result of drinking model peach beverage 1, the fresh flavor felt during eating peaches was insufficient.

### <Preparation of Second Flavor Composition (Peach Flavor B)>

Formulation value of peach flavor B was adjusted in order to bring the detection rate 1 close to the detection rate t. A calibration curve was created based on the internal standard ratio value of retronasal aroma during drinking model peach beverage 1 and the formulation value of peach flavor A. Then, using it, the formulation value of each aroma compound of peach flavor B was calculated based on the internal standard ratio during eating peaches. Formulation value of aroma compound detected only by GC/O because of evaluated that the odor intensity of retronasal aroma during drinking model peach beverage 1 and retronasal aroma during eating peaches was equivalent, was set to the same formulation value as peach flavor A. Peach flavor B was prepared by blending each aroma compound the ratio of the formulation shown in Table 3.

### <Preparation of Model Peach Beverage 2>

Model peach beverage 2 was prepared by adding peach flavor B to the above beverage base with 0.1 mass %. In addition, beverage not added flavor composition was prepared for blank measurement.

### <Collection of Exhaled Breath During Drinking Model Peach Beverage 2>

Exhaled breath during drinking model peach beverage 2 and blank beverage were collected according to the same method as in the case of model peach beverage 1.

### <Aroma Analysis by GC/MS of Exhaled Breath During Drinking Model Peach Beverage 2>

Exhaled breath during drinking model peach beverage 2 and blank beverage were analyzed by GC/MS according to the same method and analysis conditions as those for exhaled breath during drinking model peach beverage 1.

### <Aroma Analysis by GC/O of Exhaled Breath During Drinking Model Peach Beverage 2>

Exhaled breath during drinking model peach beverage 2 and blank beverage were analyzed by GC/O according to the same method and analysis conditions as those for exhaled breath during drinking model peach beverage 1. As a result, it was confirmed the presence that aroma compound not detected by GC/MS in exhaled breath during drinking model peach beverage 2 and its odor intensity was equivalent to that of exhaled breath during eating peaches.

### <Calculation of Detection Rate 2>

Detection rate 2 with the total being 100% was calculated based on an internal standard ratio of the retronasal aroma during drinking model peach beverage 2, which was calculated by the same method as in the case of the detection rate 1.

### <Detection Rate Comparison and Sensory Evaluation>

As shown in Table 3, the aroma balance was slightly different when making a comparison detection rate 2 and detection rate t.

In addition, as a result of drinking model peach beverage 2, the fresh flavor felt during eating peaches was increased as compared with model peach beverage 1, but was still insufficient.

### <Preparation of Third Flavor Composition (Peach Flavor C)>

Formulation value of peach flavor C was adjusted in order to bring the detection rate 2 close to the detection rate t. Calibration curve was created based on the internal standard ratio values of the retronasal aroma during drinking model peach beverage 1 and 2, and the formulation value of each flavor composition. Then, using it, the formulation value of each aroma compound of peach flavor C was calculated based on the internal standard ratio during eating peaches. Formulation value of aroma compound detected only by GC/O because of evaluated that the odor intensity of retronasal aroma during drinking model peach beverage 2 and retronasal aroma during eating peaches was equivalent, was set to the same formulation value as peach flavor B. Peach flavor C was prepared by blending plural aroma compounds the ratio of the formulation shown in Table 3.

### <Preparation of Model Peach Beverage 3>

Model peach beverage 3 was prepared by adding peach flavor C to the above beverage base with 0.1 mass %. In addition, beverage not added flavor composition was prepared for blank measurement.

### <Collection of Exhaled Breath During Drinking Model Peach Beverage 3>

Exhaled breath during drinking model peach beverage 3 and blank beverage were collected according to the same method as in the case of model peach beverage 1.

### <Aroma Analysis by GC/MS of Exhaled Breath During Drinking Model Peach Beverage 3>

Exhaled breath during drinking model peach beverage 3 and blank beverage were analyzed by GC/MS according to the same method and analysis conditions as those for exhaled breath during drinking model peach beverage 1.

### <Aroma Analysis by GC/O of Exhaled Breath During Drinking Model Peach Beverage 3>

Exhaled breath during drinking model peach beverage 3 and blank beverage were analyzed by GC/O according to the same method and analysis conditions as those for exhaled breath during drinking model peach beverage 1. As a result, it was confirmed the presence that aroma compound not detected by GC/MS in exhaled breath during drinking model peach beverage 3 and its odor intensity was equivalent to that of exhaled breath during eating peaches.

### <Calculation of Detection Rate 3>

Detection rate 3 with the total being 100% was calculated based on an internal standard ratio of the retronasal aroma during drinking model peach beverage 3, which was calculated by the same method as in the case of detection rate 1.

### <Detection Rate Comparison and Sensory Evaluation>

As shown in Table 3, the aroma balance was similar when making a comparison detection rate 3 and detection rate t.

In addition, as a result of drinking model peach beverage 3, the fresh and juicy flavor with a smooth fleshy texture felt during eating peaches was evoked.

### <Sensory Evaluation by Trained Panelists>

First, four trained panelists performed sensory evaluation on the flavor during eating peaches. Next, they performed sensory evaluation on the flavor during drinking model peach beverage 1 and 3. Each model peach beverage was provided to panelists in a plastic cup with a different three-digit random number written on it. Although panelists were not informed of which was model peach beverage 1 or 3, as a result, all panelists answered that model peach beverage 3 evoked the flavor felt during eating peaches as compared with model peach beverage 1.

TABLE 3
List of calculated values in flavor preparation process according to retronasal
aroma analysis during eating peaches and drinking peach beverages

| | Retronasal aroma during eating | | | Retronasal aroma during drinking | |
|---|---|---|---|---|---|
| | peaches | | | model peach beverage 1 | |
| | Internal | Detection | Peach | Internal | Detection |
| Aroma | standard | rate t | flavor A | standard | rate 1 |
| compounds | ratio | (%) | Formulation | ratio | (%) |
| Peach-1 | 14,073 | 37 | 3.7 | 95,218 | 39 |
| Peach-2 | 4,698 | 12 | 1.2 | 50,424 | 21 |
| Peach-3 | 4,212 | 11 | 1.1 | 35,009 | 14 |
| Peach-4 | 3,650 | 9.5 | 1.0 | 27,449 | 11 |
| Peach-5 | 2,515 | 6.5 | 0.66 | 17,471 | 7.2 |
| Peach-6 | 2,501 | 6.5 | 0.65 | 1,126 | 0.46 |
| Peach-7 | 1,604 | 4.2 | 0.42 | 2,222 | 0.92 |
| Peach-8 | 1,372 | 3.6 | 0.36 | 285 | 0.12 |
| Peach-9 | 585 | 1.5 | 0.15 | 3,224 | 1.3 |
| Peach-10 | 426 | 1.1 | 0.14 | 74 | 0.031 |
| Peach-11 | 345 | 0.90 | 0.090 | 1,334 | 0.55 |
| Peach-12 | 220 | 0.57 | 0.060 | 1,758 | 0.73 |
| Peach-13 | 200 | 0.52 | 0.050 | 202 | 0.083 |
| Peach-others | 2,115 | 5.5 | 0.62 | 6,479 | 2.7 |
| Water-ethanol mixed solution | | | Remainder | | |

| | | Retronasal aroma during | | | Retronasal aroma during | |
|---|---|---|---|---|---|---|
| | | drinking model peach | | | drinking model peach | |
| | | beverage 2 | | | beverage 3 | |
| | Peach | Internal | Detection | Peach | Internal | Detection |
| Aroma | flavor B | standard | rate 2 | flavor C | standard | rate 3 |
| compounds | Formulation | ratio | (%) | Formulation | ratio | (%) |
| Peach-1 | 0.28 | 11,690 | 39 | 0.26 | 4,495 | 37 |
| Peach-2 | 0.060 | 3,881 | 13 | 0.056 | 1,467 | 12 |
| Peach-3 | 0.065 | 2,778 | 9.2 | 0.072 | 1,111 | 9.0 |
| Peach-4 | 0.070 | 2,739 | 9.1 | 0.072 | 1,479 | 12 |
| Peach-5 | 0.050 | 2,373 | 7.8 | 0.044 | 676 | 5.5 |
| Peach-6 | 0.70 | 1,897 | 6.3 | 0.80 | 968 | 7.9 |
| Peach-7 | 0.15 | 1,463 | 4.8 | 0.13 | 493 | 4.0 |
| Peach-8 | 0.80 | 699 | 2.3 | 1.4 | 529 | 4.3 |
| Peach-9 | 0.014 | 759 | 2.5 | 0.0080 | 136 | 1.1 |
| Peach-10 | 0.40 | 240 | 0.79 | 0.60 | 84 | 0.69 |
| Peach-11 | 0.012 | 186 | 0.62 | 0.014 | 73 | 0.59 |
| Peach-12 | 0.0040 | 202 | 0.67 | 0.0032 | 106 | 0.86 |
| Peach-13 | 0.025 | 75 | 0.25 | 0.020 | 58 | 0.47 |
| Peach-others | 0.72 | 1,262 | 4.2 | 0.63 | 608 | 4.9 |
| Water-ethanol mixed | Remainder | | | Remainder | | |
| solution | | | | | | |

### | Example 4| Production of Strawberry-Like Flavor Composition for Beverages

### <Collection of Exhaled Breath During Eating Straw Berries>

Exhaled breath during eating strawberries was collected by the same method for apples.

### <Aroma Analysis by GC/MS of Exhaled Breath During Eating Strawberries>

Exhaled breath during eating strawberries and exhaled breath of blank was analyzed according to the same method and analysis conditions as those for exhaled breath during eating apples.

### <Aroma Analysis by GC/O of Exhaled Breath During Eating Strawberries>

Exhaled breath during eating strawberries and exhaled breath of blank were analyzed by GC/O according to the same method and analysis conditions as those for exhaled breath during eating apples. As a result, it was confirmed the presence and its odor intensity of compound that was not detected by GC/MS in exhaled breath during eating strawberries.

### <Preparation of First Flavor Composition (Strawberry Flavor A)>

An internal standard ratio of retronasal aroma during eating strawberries was calculated by the same method as in the case of apples, and then, the detection rate t with the total being 100% was calculated based on the internal standard ratio. Thereafter, the detection rate t was converted to mass %, the compound detected only by GC/O were added thereto, and the mixture was further appropriately diluted with a solvent to obtain a formulation of first flavor composition (strawberry flavor A). Formulation value of the compound detected only by GC/O was the same as the lowest formulation value among the compound detected by GC/MS. Strawberry flavor A was prepared by blending plural aroma compounds the ratio of the formulation (not described in Table 4).

### <Preparation of Model Strawberry Beverage 1>

The sugar and organic acid analysis of commercially available strawberries purchased in the season was performed, and a beverage base having an adjusted the ratio of sugar and organic acid was prepared based on the result. Though model strawberry beverage 1 was prepared by adding strawberry flavor A to the beverage base with 0.1 mass %, it had a strong smell of sulfur-containing compound and was unsuitable for drinking.

### <Preparation of Model Strawberry Beverage 2>

For the above reason, second flavor composition (strawberry flavor B) was prepared by blending plural aroma compounds the ratio of the formulation shown in Table 4, in which the amount of sulfur-containing compound was reduced from that of strawberry flavor A. Model strawberry beverage 2 was prepared by adding strawberry flavor B to the above beverage base with 0.1 mass %. In addition, beverage not added flavor composition was prepared for blank measurement.

### <Collection of Exhaled Breath During Drinking Model Strawberry Beverage 2>

Exhaled breath during drinking model strawberry beverage 2 and blank beverage were collected according to the same method as in the case of model grape beverage 1.

<Aroma Analysis by GC/MS of Exhaled Breath During Drinking Model Strawberry beverage 2>

Exhaled breath during drinking model strawberry beverage 2 and blank beverage were analyzed by GC/MS according to the same method and analysis conditions as those for exhaled breath during eating strawberries.

### <Aroma Analysis by GC/O of Exhaled Breath During Drinking Model Strawberry

### Beverage 2>

Exhaled breath during drinking model strawberry beverage 2 and blank beverage were analyzed by GC/O according to the same method and analysis conditions as those for exhaled breath during eating strawberries. As a result, it was not confirmed the presence of compound not detected by GC/MS in exhaled breath during drinking model strawberry beverage 2.

### <Calculation of Detection Rate 2>

Detection rate 2 with the total being 100% was calculated based on an internal standard ratio of the retronasal aroma during drinking model strawberry beverage 2, which was calculated by the same method as in <Preparation of first flavor composition (strawberry flavor A)>.

### <Detection Rate Comparison and Sensory Evaluation>

As shown in Table 4, the aroma balance was significantly different when the detection rate t of the retronasal aroma during eating strawberries was compared with the detection rate 2 of the retronasal aroma during drinking model strawberry beverage 2.

In addition, as a result of drinking model strawberry beverage 2, the fresh flavor felt during eating strawberries was insufficient.

### <Preparation of Third Flavor Composition (Strawberry Flavor C)>

Formulation value of strawberry flavor C was adjusted in order to bring the detection rate 2 close to the detection rate t. Calibration curve was created based on the internal standard ratio value of the retronasal aroma during drinking model strawberry beverage 2 and the formulation value of strawberry flavor B. Then, using it, the formulation value of each aroma compound of strawberry flavor C was calculated based on the internal standard ratio value of the retronasal aroma during eating strawberries. Formulation value of aroma compound detected only by GC/O was increased compared to strawberry flavor B. It was because the amount of that aroma compound in exhaled breath during drinking strawberry beverage 2 was lower than that in exhaled breath during eating strawberries. Strawberry flavor C was prepared by blending plural aroma compounds the ratio of the formulation shown in Table 4.

### <Preparation of Model Strawberry Beverage 3>

Model strawberry beverage 3 was prepared by adding strawberry flavor C to the above beverage base with 0.1 mass %. In addition, beverage not added flavor composition was prepared for a blank measurement.

### <Collection of Exhaled Breath During Drinking Model Strawberry Beverage 3>

Exhaled breath during drinking model strawberry beverage 3 and blank beverage were collected according to the same method as in the case of model strawberry beverage 2.

### <Aroma Analysis by GC/MS of Exhaled Breath During Drinking Model Strawberry Beverage 3>

Exhaled breath during drinking model strawberry beverage 3 and blank beverage were analyzed by GC/MS according to the same method and analysis conditions as those for exhaled breath during drinking model strawberry beverage 2.

### <Aroma Analysis by GC/O of Exhaled Breath During Drinking Model Strawberry Beverage 3>

Exhaled breath during drinking model strawberry beverage 3 and blank beverage were analyzed by GC/O according to the same method and analysis conditions as those for exhaled breath during drinking model strawberry beverage 2. As a result, it was confirmed the presence that aroma compound not detected by GC/MS in exhaled breath during drinking model strawberry beverage 3 and its odor intensity was equivalent to that of exhaled breath during eating strawberries.

### <Calculation of Detection Rate 3>

Detection rate 3 with the total being 100% was calculated based on an internal standard ratio of the retronasal aroma during drinking model strawberry beverage 3, which was calculated by the same method as in the case of detection rate 2.

### <Detection Rate Comparison and Sensory Evaluation>

As shown in Table 4, the aroma balance was slightly different when making a comparison detection rate 3 and detection rate t.

In addition, as a result of drinking model strawberry beverage 3, the fresh flavor along with sweetness felt during eating strawberries was increased as compared with model strawberry beverage 2, but was still insufficient.

### <Preparation of Fourth Flavor Composition (Strawberry Flavor D)>

Formulation value of strawberry flavor D was adjusted in order to bring the detection rate 3 close to the detection rate t. Calibration curve was created based on these internal standard ratio values of the retronasal aroma during drinking model strawberry beverage 2 and 3, and the formulation value of each flavor composition. Then, using it, the formulation value of each aroma compound of strawberry flavor D was calculated based on the internal standard ratio value of the retronasal aroma during eating strawberries. Formulation value of aroma compound detected only by GC/O, because of evaluated that the odor intensity of retronasal aroma during drinking model strawberry beverage 2 and retronasal aroma during eating strawberries was equivalent, was set to the same formulation value as strawberry flavor C. Strawberry flavor D was prepared by blending plural aroma compounds the ratio of the formulation shown in Table 4.

### <Preparation of Model Strawberry Beverage 4>

Model strawberry beverage 4 was prepared by adding strawberry flavor D to the above beverage base with 0.1 mass %. In addition, beverage not added flavor composition was prepared for blank measurement.

### <Collection of Exhaled Breath During Drinking Model Strawberry Beverage 4>

Exhaled breath during drinking model strawberry beverage 4 and blank beverage were collected according to the same method as in the case of model strawberry beverage 2.

### <Aroma Analysis by GC/MS of Exhaled Breath During Drinking Model Strawberry Beverage 4>

Exhaled breath during drinking model strawberry beverage 4 and blank beverage were analyzed by GC/MS according to the same method and analysis conditions as those for exhaled breath during drinking model strawberry beverage 2.

### <Aroma Analysis by GC/O of Exhaled Breath During Drinking Model Strawberry Beverage 4>

Exhaled breath during drinking the model strawberry beverage 4 and the blank beverage were analyzed by GC/O according to the same method and analysis conditions as those for exhaled breath during drinking model strawberry beverage 2. As a result, it was confirmed the presence that aroma compound not detected by GC/MS in exhaled breath during drinking model strawberry beverage 4 and its odor intensity was equivalent to that of exhaled breath during eating strawberries.

### <Calculation of Detection Rate 4>

Detection rate 4 with the total being 100% was calculated based on an internal standard ratio of the retronasal aroma during drinking model strawberry beverage 4, which was calculated by the same method as in the case of detection rate 2.

### <Detection Rate Comparison and Sensory Evaluation>

As shown in Table 4, the aroma balance was similar when making a comparison detection rate 4 and detection rate t.

In addition, as a result of drinking model strawberry beverage 4, the fresh and juicy flavor with sweetness felt during eating strawberries was evoked.

### <Sensory Evaluation by Trained Panelists>

First, four trained panelists performed sensory evaluation on the flavor during eating strawberries. Next, they performed sensory evaluation on the flavor during drinking model strawberry beverage 2 and 4. Each model strawberry beverage was provided to panelists in a plastic cup with a different three-digit random number written on it. Although panelists were not informed of which was model strawberry beverage 2 or 4, as a result, all panelists answered that model strawberry beverage 4 evoked the flavor felt during eating strawberries as compared with model strawberry beverage 2.

TABLE 4
List of calculated values in flavor preparation process according to retronasal
aroma analysis during eating strawberries and drinking strawberry beverages

| | |
|---|---|
| Retronasal aroma during eating | Retronasal aroma during drinking |
| strawberries | model strawberry beverage 2 |

| | Internal | Detection | Strawberry | Internal | Detection |
|---|---|---|---|---|---|
| Aroma | standard | rate t | flavor B | standard | rate 2 |
| compounds | ratio | (%) | Formulation | ratio | (%) |
| Strawberry-1 | 2,445 | 31 | 0.80 | 17,206 | 8.3 |
| Strawberry-2 | 1,422 | 18 | 4.0 | 126,986 | 61 |
| Strawberry-3 | 696 | 8.9 | 0.16 | 2,317 | 1.1 |
| Strawberry-4 | 467 | 6.0 | 0.24 | 1,551 | 0.74 |
| Strawberry-5 | 460 | 5.9 | 0.080 | 888 | 0.43 |
| Strawberry-6 | 412 | 5.3 | 0.16 | 5,126 | 2.5 |
| Strawberry-7 | 410 | 5.2 | 0.16 | 7,077 | 3.4 |
| Strawberry-8 | 407 | 5.2 | 0.080 | 1,700 | 0.82 |
| Strawberry-9 | 248 | 3.2 | 0.080 | 2,449 | 1.2 |
| Strawberry-10 | 188 | 2.4 | 0.080 | 3,512 | 1.7 |
| Strawberry-11 | 151 | 1.9 | 0.80 | 28,467 | 14 |
| Strawberry-12 | 140 | 1.8 | 1.6 | 730 | 0.35 |
| Strawberry-13 | 83 | 1.1 | 0.24 | 8,557 | 4.1 |
| Strawberry-14 | 75 | 0.95 | 0.0080 | 139 | 0.067 |
| Strawberry-15 | 23 | 0.29 | 0.0016 | 143 | 0.069 |
| Strawberry-others | 206 | 2.6 | 5.0 | 1,420 | 0.68 |
| Water-ethanol mixed | | | Remainder | | |
| solution | | | | | |

| | | Retronasal aroma during | | | Retronasal aroma during | |
|---|---|---|---|---|---|---|
| | | drinking model strawberry | | | drinking model strawberry | |
| | | beverage 3 | | | beverage 4 | |
| | Strawberry | Internal | Detection | Strawberry | Internal | Detection |
| Aroma | flavor C | standard | rate 1 | flavor D | standard | rate 3 |
| compounds | Formulation | ratio | (%) | Formulation | ratio | (%) |
| Strawberry-1 | 0.80 | 11,966 | 31 | 0.80 | 9,747 | 30 |
| Strawberry-2 | 0.40 | 10,972 | 28 | 0.28 | 6,488 | 20 |
| Strawberry-3 | 0.40 | 1,952 | 5.1 | 0.32 | 2,864 | 8.8 |
| Strawberry-4 | 0.50 | 2,128 | 5.5 | 0.18 | 1,790 | 5.5 |
| Strawberry-5 | 0.20 | 2,032 | 5.3 | 0.25 | 2,228 | 6.9 |
| Strawberry-6 | 0.080 | 1,797 | 4.7 | 0.090 | 1,839 | 5.7 |
| Strawberry-7 | 0.060 | 1,852 | 4.8 | 0.080 | 2,083 | 6.4 |
| Strawberry-8 | 0.020 | 242 | 0.6 | 0.16 | 1,738 | 5.4 |
| Strawberry-9 | 0.060 | 1,276 | 3.3 | 0.080 | 1,124 | 3.5 |
| Strawberry-10 | 0.040 | 1,179 | 3.1 | 0.10 | 728 | 2.2 |
| Strawberry-11 | 0.080 | 1,052 | 2.7 | 0.030 | 679 | 2.1 |
| Strawberry-12 | 2.0 | 511 | 1.3 | 1.500 | 367 | 1.1 |
| Strawberry-13 | 0.020 | 582 | 1.5 | 0.0 | 325 | 1.0 |
| Strawberry-14 | 0.020 | 171 | 0.44 | 0.040 | 282 | 0.87 |
| Strawberry-15 | 0.0020 | 117 | 0.30 | 0.0020 | 75 | 0.23 |
| Strawberry-others | 3.7 | 764 | 2.3 | 2.6 | 41 | 0.13 |
| Water-ethanol mixed | Remainder | | | Remainder | | |
| solution | | | | | | |

### [Example 5] Production of Strawberry Flavor Composition for Gummy Candies

The detection rate t of the retronasal aroma during eating strawberries was obtained by the method shown in Example 4 (production of a strawberry flavor composition for beverages).

### <Preparation of First Flavor Composition (Strawberry Flavor E)>

Formulation value of strawberry flavor E was based on strawberry flavor D, which was prepared for the strawberry beverage, with the overall strength enhanced. Strawberry flavor E was prepared by blending plural aroma compounds the ratio of the formulation shown in Table 5.

### <Preparation of Model Strawberry Gummy Candy 1>

Model strawberry gummy candy 1 was prepared by adding strawberry flavor E to general gummy candy base with 0.2 mass %. In addition, gummy candy not added flavor composition was prepared for blank measurement.

### <Collection of Exhaled Breath During Eating Model Strawberry Gummy Candy 1>

Exhaled breath during eating model strawberry gummy candy 1 and blank gummy candy were collected according to the same method as in the case of eating strawberries.

### <Aroma Analysis by GC/MS of Exhaled Breath During Eating Model Strawberry Gummy Candy 1>

Exhaled breath during eating model strawberry gummy candy 1 and blank gummy candy were analyzed by GC/MS according to the same method and analysis conditions as those for exhaled breath during eating strawberries.

### <Aroma Analysis by GC/O of Exhaled Breath During Eating Model Strawberry Gummy Candy 1>

Exhaled breath during eating model strawberry gummy candy 1 and blank gummy candy were analyzed by GC/O according to the same method and analysis conditions as those for exhaled breath during eating strawberries. As a result, it was confirmed that the presence of aroma compound that was not detected by GC/MS in exhaled breath during eating model strawberry gummy candy 1. And its odor intensity was stronger than that of exhaled breath during eating strawberries.

### <Calculation of the Detection Rate 1>

The detection rate 1 with the total being 100% was calculated based on an internal standard ratio of the retronasal aroma during eating the model strawberry gummy 1, which was calculated by the same method as in the case of the exhaled breath during eating strawberries.

### <Detection Rate Comparison and Sensory Evaluation>

As shown in Table 5, the aroma balance was significantly different when the detection rate t of the retronasal aroma during eating strawberries was compared with the detection rate 1 of the retronasal aroma during eating the model strawberry gummy candy 1.

In addition, as a result of eating the model strawberry gummy candy 1, the fresh flavor felt during eating strawberries was insufficient.

### <Preparation of Second Flavor Composition (Strawberry Flavor F)>

Formulation value of strawberry flavor F was adjusted in order to bring the detection rate 1 close to the detection rate t. Calibration curve was created based on the internal standard ratio value of the retronasal aroma during eating the model strawberry gummy candy 1 and the formulation value of strawberry flavor E. Then, using it, the formulation value of each aroma compound of strawberry flavor F was calculated based on the internal standard ratio value of the retronasal aroma during eating strawberries. Formulation value of aroma compound detected only by GC/O was decreased compared to strawberry flavor E. It was because the amount of aroma compound in exhaled breath during eating gummy candy 1 was higher than that in exhaled breath during eating strawberries. Strawberry flavor F was prepared by blending plural aroma compound the ratio of the formulation shown in Table 5.

### <Preparation of Model Strawberry Gummy Candy 2>

Model strawberry gummy candy 2 was prepared by adding strawberry flavor F to the above gummy candy base with 0.2 mass %. In addition, a gummy candy not added flavor composition was prepared for blank measurement.

### <Collection of Exhaled Breath During Eating Model Strawberry Gummy Candy 2>

Exhaled breath during eating model strawberry gummy candy 2 and blank gummy candy were collected according to the same method as in the case of the model strawberry gummy candy 1.

### <Aroma Analysis by GC/MS of Exhaled Breath During Eating Model Strawberry Gummy Candy 2>

Exhaled breath during eating model strawberry gummy candy 2 and blank gummy candy were analyzed by GC/MS according to the same method and analysis conditions as those for exhaled breath during eating model strawberry gummy candy 1.

### <Aroma Analysis by GC/O of Exhaled Breath During Eating Model Strawberry Gummy Candy 2>

Exhaled breath during eating model strawberry gummy candy 2 and blank gummy candy were analyzed by GC/O according to the same method and analysis conditions as those for exhaled breath during eating model strawberry gummy candy 1. As a result, it was confirmed the presence that aroma compound not detected by GC/MS in exhaled breath during eating model strawberry gummy 2 and its odor intensity was equivalent to that of exhaled breath during eating strawberries.

### <Calculation of Detection Rate 2>

Detection rate 2 with the total being 100% was calculated based on an internal standard ratio of the retronasal aroma during eating model strawberry gummy candy 2, which was calculated by the same method as in the case of detection rate 1.

### <Detection Rate Comparison and Sensory Evaluation>

As shown in Table 5, the aroma balance was slightly different when making a comparison detection rate 2 and detection rate t.

In addition, as a result of eating model strawberry gummy candy 2, the fresh flavor along with sweetness felt during eating strawberries was increased as compared with model strawberry gummy candy 1, but was still insufficient.

### <Preparation of Third Flavor Composition (Strawberry Flavor G)>

Formulation value of strawberry flavor D was adjusted in order to bring the detection rate 2 close to the detection rate t. Calibration curve was created based on these internal standard ratio values of the retronasal aroma during eating model strawberry gummy candy 1 and 2, and the formulation value of each flavor composition. Then, using it, the formulation value of each aroma compound was calculated based on the internal standard ratio of the retronasal aroma during eating strawberries. Formulation value of aroma compound detected only by GC/O, because of evaluated that the odor intensity of retronasal aroma during eating strawberry gummy candy 2 and retronasal aroma during eating strawberries was equivalent, was set to the same formulation value as strawberry flavor F. Third flavor composition (strawberry flavor G) was prepared by blending plural aroma compounds the ratio of the formulation shown in Table 5.

### <Preparation of Model Strawberry Gummy Candy 3>

Model strawberry gummy candy 3 was prepared by adding strawberry flavor G to above gummy candy base with 0.2 mass %. In addition, gummy candy not added flavor composition was prepared for blank measurement.

### <Collection of Exhaled Breath During Eating Model Strawberry Gummy Candy 3>

Exhaled breath during eating the model strawberry gummy candy 3 and blank gummy candy were collected according to the same method as in the case of the model strawberry gummy candy 1.

### <Aroma Analysis by GC/MS of Exhaled Breath During Eating Model Strawberry Gummy Candy 3>

Exhaled breath during eating model strawberry gummy candy 3 and a blank gummy candy were analyzed by GC/MS according to the same method and analysis conditions as those for the exhaled breath during eating the model strawberry gummy candy 1.

### <Calculation of Detection Rate 3>

Detection rate 3 with the total being 100% was calculated based on an internal standard ratio of the retronasal aroma during eating the model strawberry gummy candy 3, which was calculated by the same method as in case of the detection rate 1.

### <Aroma Analysis by GC/O of Exhaled Breath During Eating Model Strawberry Gummy Candy 3>

Exhaled breath during eating the model strawberry gummy candy 3 and blank gummy candy were analyzed by GC/O according to the same method and analysis conditions as those for exhaled breath during eating model strawberry gummy candy 1. As a result, it was confirmed the presence that aroma compound not detected by GC/MS in exhaled breath during eating model strawberry gummy candy 3 and its odor intensity was equivalent to that of the exhaled breath during eating strawberries.

### <Detection Rate Comparison and Sensory Evaluation>

As shown in Table 5, the aroma balance was similar when making a comparison detection rate 3 and detection rate t.

In addition, as a result of eating model strawberry gummy candy 3, the fresh and juicy flavor with sweetness felt during eating strawberries was evoked.

### <Sensory Evaluation by Trained Panelists>

First, four trained panelists performed sensory evaluation on the flavor during eating strawberries. Next, they performed sensory evaluation the flavor during eating model strawberry gummy candy 1 and model strawberry gummy candy 3. Each model strawberry gummy candy was provided to panelists in a plastic cap with a different three-digit random number written on it. Although panelists were not informed of which was model strawberry gummy candy 1 or 3, as a result, all panelists answered that model strawberry gummy candy 3 evoked the flavor felt during eating strawberries as compared with model strawberry gummy candy 1.

TABLE 5
List of calculated values in flavor preparation process according to retronasal
aroma analysis during eating strawberries and eating strawberry gummy candies

| | Retronasal aroma during eating | | | Retronasal aroma during eating | |
|---|---|---|---|---|---|
| | strawberries | | | strawberry gummy candy 1 | |
| | Internal | Detection | Strawberry | Internal | Detection |
| Aroma | standard | rate t | flavor E | standard | rate 1 |
| compounds | ratio | (%) | Formulation | ratio | (%) |
| Strawberry-1 | 2,445 | 31 | 3.2 | 16,122 | 24 |
| Strawberry-2 | 1,422 | 18 | 1.1 | 15,315 | 23 |
| Strawberry-3 | 696 | 8.9 | 1.3 | 11,175 | 17 |
| Strawberry-4 | 467 | 6.0 | 0.72 | 402 | 0.61 |
| Strawberry-5 | 460 | 5.9 | 1.0 | 854 | 1.3 |
| Strawberry-6 | 412 | 5.3 | 0.36 | 4,964 | 7.5 |
| Strawberry-7 | 410 | 5.2 | 0.32 | 5,289 | 8.0 |
| Strawberry-8 | 407 | 5.2 | 0.64 | 3,645 | 5.5 |
| Strawberry-9 | 248 | 3.2 | 0.32 | 1,932 | 2.9 |
| Strawberry-10 | 188 | 2.4 | 0.40 | 1,835 | 2.8 |
| Strawberry-11 | 151 | 1.9 | 0.12 | 1,431 | 2.2 |
| Strawberry-12 | 140 | 1.8 | 6.0 | 1,434 | 2.2 |
| Strawberry-13 | 83 | 1.1 | 0.060 | 750 | 1.1 |
| Strawberry-14 | 75 | 0.95 | 0.16 | 393 | 0.60 |
| Strawberry-15 | 23 | 0.29 | 0.0080 | 75 | 0.11 |
| Strawberry-others | 206 | 2.6 | 8.2 | 432 | 0.65 |
| Propylene glycol | | | Remainder | | |

| | | Retronasal aroma during | | | Retronasal aroma during | |
|---|---|---|---|---|---|---|
| | | eating strawberry gummy | | | eating strawberry gummy | |
| | | candy 2 | | | candy 3 | |
| | Strawberry | Internal | Detection | Strawberry | Internal | Detection |
| Aroma | flavor F | standard | rate 2 | flavor G | standard | rate 3 |
| compounds | Formulation | ratio | (%) | Formulation | ratio | (%) |
| Strawberry-1 | 2.5 | 38,964 | 39 | 1.6 | 47,607 | 29 |
| Strawberry-2 | 0.50 | 16,495 | 16 | 0.40 | 27,928 | 17 |
| Strawberry-3 | 0.50 | 8,160 | 8.2 | 0.40 | 12,894 | 7.9 |
| Strawberry-4 | 1.8 | 3,066 | 3.1 | 2.6 | 23,144 | 14 |
| Strawberry-5 | 1.0 | 4,893 | 4.9 | 0.96 | 7,320 | 4.5 |
| Strawberry-6 | 0.15 | 4,214 | 4.2 | 0.15 | 9,188 | 5.6 |
| Strawberry-7 | 0.13 | 6,065 | 6.1 | 0.080 | 6,072 | 3.7 |
| Strawberry-8 | 0.35 | 7,120 | 7.1 | 0.20 | 8,016 | 4.9 |
| Strawberry-9 | 0.15 | 3,226 | 3.2 | 0.12 | 5,393 | 3.3 |
| Strawberry-10 | 0.050 | 1,624 | 1.6 | 0.060 | 4,378 | 2.7 |
| Strawberry-11 | 0.050 | 1,630 | 1.6 | 0.048 | 3,110 | 1.9 |
| Strawberry-12 | 3.0 | 1,363 | 1.4 | 3.0 | 2,860 | 1.8 |
| Strawberry-13 | 0.035 | 1,019 | 1.0 | 0.030 | 1,953 | 1.2 |
| Strawberry-14 | 0.15 | 1,354 | 1.4 | 0.080 | 1,399 | 0.86 |
| Strawberry-15 | 0.0050 | 181 | 0.18 | 0.0048 | 424 | 0.26 |
| Strawberry-others | 3.9 | 639 | 0.64 | 4.1 | 1,283 | 0.79 |
| Propylene glycol | Remainder | | | Remainder | | |

Although the present invention has been described in detail and with reference to specific embodiments, it will be apparent to those skilled in the art that various changes and modifications can be made without departing from the spirit and scope of the present invention. This application is based on a Japanese Patent Application (No. 2022-067737) filed on Apr. 15, 2022, the contents of which are incorporated herein as a reference.

1. A method for producing a flavor composition, the method comprising:
a step (1) of analyzing retronasal aroma during eating or drinking an analyte by gas chromatography;
a step (2) of preparing a first flavor composition containing aroma compounds, by calculating a detection rate t of these plural aroma compounds in the retronasal aroma of the step (1) based on a result of the gas chromatography analysis of the step (1);
a step (3) of preparing a model food or beverage 1 by adding the first flavor composition;
a step (4) of analyzing retronasal aroma during eating or drinking the model food or beverage 1 by gas chromatography;
a step (5) of calculating a detection rate 1 of plural aroma compounds in the retronasal aroma of the step (4) based on a result of the gas chromatography analysis of the step (4);
a step (6) of preparing a second flavor composition by adjusting an aroma balance of the first flavor composition to bring the detection rate 1 close to the detection rate t; and
a step (7) of preparing a final flavor composition having an aroma balance matching the detection rate t by repeating the same operations as the steps (3) to (6).

2. The method for producing a flavor composition according to claim 1, wherein the step (1) includes a step of analyzing the retronasal aroma by gas chromatography/olfactometry then adding the detected aroma compound to first flavor composition.

3. The method for producing a flavor composition according to claim 2, wherein the gas chromatography/olfactometry analysis includes of adjusting an odor intensity by using an aroma extract dilution analysis method.

4. The method for producing a flavor composition according to claim 1, wherein the steps (1) and (4) each includes a step of adsorbing the retronasal aroma to an adsorbent and desorbing the adsorbed retronasal aroma.

5. The method for producing a flavor composition according to claim 2, wherein the steps (1) and (4) each includes a step of adsorbing the retronasal aroma to an adsorbent and desorbing the adsorbed retronasal aroma.

6. The method for producing a flavor composition according to claim 3, wherein the steps (1) and (4) each includes a step of adsorbing the retronasal aroma to an adsorbent and desorbing the adsorbed retronasal aroma.
